(19) United States
(12) Patent Application Publication
(10) Pub. No.: US 2020/0038323 A1
(43) Pub. Date: Feb. 6, 2020

(54) OPTICALLY CLEAR, IN-SITU FORMING BIODEGRADABLE NANO-CARRIERS FOR OCULAR THERAPY, AND METHODS USING SAME

(71) Applicant: Rowan University, Glassboro, NJ (US)

(72) Inventors: Mark E. Byrne, Glassboro, NJ (US); Mindy George-Weinstein, Glassboro, NJ (US); Laura L. Osorno, Glassboro, NJ (US)

(21) Appl. No.: 16/498,689

(22) PCT Filed: Mar. 31, 2018
(86) PCT No.: PCT/US18/25604

§ 371 Date: Sep. 27, 2019

Related U.S. Application Data
(60) Provisional application No. 62/479,716, filed on Mar. 31, 2017.

(51) Int. Cl. A61K 9/107; A61K 9/00; A61K 31/573; A61K 31/728

(52) U.S. Cl. CPC A61K 9/1075; A61K 31/728; A61K 31/573; A61K 9/0048

(57) ABSTRACT

In one aspect, the present invention relates to thermo-reversible hydrogel drug delivery compositions comprising at least one biodegradable copolymer drug carrier. In certain embodiments, the hydrogel compositions of the invention are optically clear and suitable for use in local delivery of ocular therapeutics. In other embodiments, the hydrogel compositions of the invention provide a means for controlled or sustained drug delivery.

### CROSS-REFERENCE TO RELATED APPLICATIONS

This application claims priority under 35 U.S.C. § 119(e) to U.S. Provisional Application No. 62/479,716, filed Mar. 31, 2017, which is incorporated herein by reference in its entirety.

### BACKGROUND OF THE INVENTION

The eye is a one of the most complex and protected organs in the human body. Anatomically, the anterior segment is composed of the cornea, conjunctiva, aqueous humor, iris, ciliary body, and the lens. The posterior segment includes the sclera, choroid, retinal pigment epithelium, neural retina, optic nerve, and vitreous humor. Some of the most common, vision-threatening diseases affecting the anterior segment include, but are not limited to, glaucoma, allergic conjunctivitis, cataract, secondary cataract or posterior capsule opacification, and anterior uveitis. However, diseases that typically affect the posterior segment are macular degeneration disease and diabetic retinopathy.

The physiological and anatomical complexity of the eye makes it a highly protected organ, substantially limiting drug delivery. Topically applied drugs, like eye drops, are the most widely used non-invasive routes of drug administration to treat anterior segment diseases. Eye drops make up over 90% of the marketed ophthalmic formulations because of the ease of administration and patient compliance. However, the physiological and anatomical complexity of the eye act as a barrier that impedes drug permeation, making the drug bioavailability and therapeutic concentrations low. Very small amounts, if any, of the topical active ingredient in the applied dose reaches the deep ocular tissues, including the posterior segment of the eye. To reach the posterior segment, intravitreal injections and periocular injections are the recommended routes of drug administration. Nonetheless, the need of repeated eye puncture causes several side effects such as endophthalmitis, hemorrhage, retinal detachment, and poor patient tolerance. Hence, there is a wide need of designing more efficient ocular drug delivery systems that provide sustained administration of a therapeutic.

Posterior Capsule Opacification (PCO) is a vision impairing condition that arises in 20% of adult patients and nearly all children within the first 3 years following primary cataract surgery. The fibrotic form of PCO, which is more detrimental to vision, is characterized by the appearance of wrinkles in the capsule surrounding the lens, and aggregates of differentiating lens epithelial cells.

Historically, the most successful treatment for PCO is the use of neodymium-doped yttrium aluminum garnet; Nd:Y₃Al₅O₁₂ (Nd:YAG)) laser capsulotomy. This therapeutic surgery clears the visual axis and restores vision by disrupting ocular tissue. The estimated cost to Medicare for Nd:YAG laser surgery is $250 million dollars annually in the US. However, such therapy is not available worldwide and complications can arise including retinal detachment, damage to the intraocular lens, and glaucoma. Because of the expensive nature of the treatment plan and rare accessibility to it, convenient and cost effective treatments for PCO remains an unmet need in ophthalmology.

Biodegradable polymers such as PLGA copolymers and poloxamers (PEO—PPO-PEO) have been used as surgical sutures, wound dressings, and drug delivery systems. However, PEG-PLGA-PEG triblock copolymeric microsphere and nanosphere systems have generally been disfavored for therapeutic applications due to the complex manufacturing methods. In addition, poloxamers have weak mechanical strength and present toxicity at high concentrations.

There remains a need in the art for compositions and methods for more efficient and efficacious ocular drug delivery systems. In certain embodiments, such compositions and methods should be useful for diseases associated with the anterior segment of the eye, such as PCO. The present invention meets and addresses these needs.

### BRIEF SUMMARY OF THE INVENTION

In one aspect, the invention provides a composition comprising a biodegradable copolymer comprising an A-B-A block structure. In another aspect, the invention provides a method of treating or preventing a disease or disorder in the eye of a subject in need thereof, the method comprising administering to the subject a therapeutically effective amount of a composition of the invention.

In certain embodiments, the A block is at least one selected from the group consisting of poly(D,L-lactic-co-glycolic acid) (PLGA), poly(propylene oxide) (PPO), poly(dioxanone) (PDS), and poly(L-lactic acid-co-caprolactone) (PLLACL). In other embodiments, the B block is at least one selected from the group consisting of polyethylene glycol (PEG), poly(vinyl alcohol) (PVA), hydroxypropyl methylcellulose (HPMC), poly(2-hydroxyethyl methacrylate) (polyHEMA), chitosan, and methoxy poly(ethylene glycol) (MPEG). In yet other embodiments, the composition further comprises at least one pharmaceutical agent. In yet other embodiments, the composition further comprises at least one pharmaceutically acceptable carrier. In yet other embodiments, the composition has a gelation temperature (GT) of around 30° C. to about 37° C. In yet other embodiments, the composition has a GT of around 34° C. to about 37° C.

In certain embodiments, the biodegradable copolymer has an average molecular weight (MW) of about 1,000 to about 20,000 Daltons.

In certain embodiments, the A block is PLGA. In other embodiments, the B block is PEG. In yet other embodiments, the A block is PLGA and the B block is PEG. In yet other embodiments, the PEG component of the copolymer has an average MW of about 500 to about 2,500 Daltons. In yet other embodiments, the weight ratio of the A block to the B block is about 1/1 to about 5/1. In yet other embodiments, the weight ratio of PLGA to PEG (PLGA wt/PEG wt) is about 1/1 to about 5/1. In yet other embodiments, the A block/B block weight ratio is at least one selected from the group consisting of about 5/1, 3/1, 2.3/1, 2/1, 1.5/1, and 1/1. In yet other embodiments, the PLGA/PEG weight ratio is at least one selected from the group consisting of about 5/1, 3/1, 2.3/1, 2/1, 1.5/1, and 1/1. In yet other embodiments, the PLGA component of the copolymer comprises a (D,L)-lactic acid/glycolic acid (LA/GA) molar ratio of about 1/1 to about 35/1. In yet other embodiments, the LA/GA molar ratio is at least one selected from the group consisting of about 35/1, 25/1, 20/1, 15/1, 12.5/1, 10/1, 7.5/1, 6/1, 5/1, 3/1, 2.5/1, 2/1, 1.5/1, and 1/1. In yet other embodiments, the PLGA component of the copolymer has an average MW of about 1,000 to about 2500 Daltons.

In certain embodiments, the composition further comprises at least one multivalent polyion. In other embodiments, the concentration of the at least one multivalent polyion in the biodegradable copolymer is about 0.1 mg/μL to about 150 mg/μL. In yet other embodiments, the concentration of the at least one multivalent polyion in the biodegradable copolymer composition is about 10 mg/μL. In yet other embodiments, the at least one multivalent polyion is at least one selected from the group consisting of poly(L-Lysine) (PLL), polyethylenimine (PEI), poly[α-aminobutyl)-1-glycolic acid] (PAGA), poly(β-amino esters) (PBAEs), Polydiallyldimethylammonium chloride (polyDADMAC), chitosan, poly(glutamic acid) (PGA), hyaluronic acid (HA), poly(alkyl cyanoacrylate), and poly(acrylic acid) (PAA). In yet other embodiments, the at least one multivalent polyion complexes at least a portion of the at least one pharmaceutical agent. In yet other embodiments, the complexed pharmaceutical agent has a lower release rate from the composition than the pharmaceutical agent in the absence of the at least one multivalent polyion.

In certain embodiments, the biodegradable copolymer has a polydispersity index of about 1.2 to about 2.0.

In certain embodiments, the pharmaceutically acceptable carrier is a buffered saline solution.

In certain embodiments, the biodegradable copolymer is present in the composition at a concentration of about 5 mg/μL to about 30 mg/μL.

In certain embodiments, the at least one pharmaceutical agent is selected from the group consisting of an antimicrobial agent, antibiotic, anti-inflammatory agent, corticosteroid, SAID, NSAID, immunosuppressive agent, immune-modulating agent, apoptosis inducing agent, anti-cancer agent, cycloplegic agent, mydriatic agent, comfort agent, lubricating agent, anti-glaucoma agent, anti-allergy agent, cytotoxic agent, anti-TNF agent, collagen, gamma-globulin, interferon, vasoconstrictor agent, vasodilation agent, platelet activator factor antagonist, plasminogen activator (tPA), streptokinase (SK), urokinase (UK), anesthetic agent, numbing agent, nitric oxide synthase inhibitor, antifungal agent, antiviral agent, antiprotozoal agent, hemolytic, antiparasitic agent, antibody, protein, carbohydrate, DNA segment, RNA segment, or any combinations thereof.

In certain embodiments, the at least one pharmaceutical agent is at least one selected from the group consisting of lapatinib, sunitinib, sorafenib, axitinib, cediranib, ranibizumab, pegaptanib pazopanib, dexamethasone, dexamethasone sodium phosphate, beta-methasone, loteprednol etabonate, ketotifen free base or any salt thereof (such as, for example, fumarate), levocabastine free base or any salt thereof (such as hydrochloride), diclofenac free acid or any salt thereof (such as sodium), bromfenac free acid or any salt thereof (such as sodium), moxifloxacin, trehalose, prednisolone, prednisolone acetate, prednisolone sodium phosphate, ibuprofen, latanoprost, doxorubicin, hyaluronic acid, fluorescein isothyocyanate-hyaluronic acid, fluorescein isothiocyanate-dextran, dextran, hydroxypropoylemethycellulose, cyclosporine, lidocaine, bupivacaine, procaine, prilocaine, mepivaaine, dibucaine, levobupivaine, cocaine, nucleic acid nanospheres, nucleic acid conjugates, nucleic acid drug conjugates, or any salts or solvates thereof.

In certain embodiments, the composition further comprises at least one visualization agent.

In certain embodiments, above the GT, the hydrogel composition forms an orderly packed, three-dimensional hydrogel. In other embodiments, the hydrogel is a substantially transparent gel. In yet other embodiments, the hydrogel has a visible light transparency of at least about 50%, 55%, 60%, 65%, 70%, 75%, 80%, 85%, 90%, 95%, 96%, 97%, 98%, 99%, 99.5%, or 100%. In other embodiments, the hydrogel has a visible light transparency of about 50% to visible light from 390 to 500 nm, and at least 75% to visible light from 500-780 nm. In yet other embodiments, the hydrogel formed in situ has a visible light transparency of at least 75%. In yet other embodiments, the hydrogel prevents transmittance of at least a fraction of ultraviolet light (and/or a fraction of a range thereof).

In certain embodiments, the disease or disorder is at least one selected from the group consisting of posterior capsule opacification (PCO), age-related macular degeneration (AMD), diabetic eye disease, diabetic macular edema (DME), macular edema, uveitis, glaucoma, Behcet's Disease (Adamantiades-Behcet's disease), blepharospasm, corneal diseases, retinal diseases, dry eye diseases, eye inflammation, eye infection, post-surgical trauma, eye infection and eye inflammation.

In certain embodiments, the composition is administered to the subject via intraocular injection.

In certain embodiments, the composition is administered to the subject at a temperature below its GT, such that the composition is capable of being administered by injection to the subject and the composition undergoes thermo-reversible gelation to form a hydrogel after administration to the subject. In other embodiments, the hydrogel formed after the composition undergoes thermo-reversible gelation transmits at least 50% of visible light. In yet other embodiments, the hydrogel formed after the composition undergoes thermo-reversible gelation allows for controlled release of at least 50% of the at least one pharmaceutical agent over a period of time selected from the group consisting of 1 day, 3 days, 7 days, 2 weeks, 1 month, 2 months, 3 months, 4 months, 6 months and 1 year.

In certain embodiments, the subject is a mammal. In other embodiments, the subject is a human.

### BRIEF DESCRIPTION OF THE DRAWINGS

The following detailed description of specific embodiments of the invention will be better understood when read in conjunction with the appended drawings. For the purpose of illustrating the invention, specific embodiments are shown in the drawings. It should be understood, however, that the invention is not limited to the precise arrangements and instrumentalities of the embodiments shown in the drawings.

FIG. 1 is a scheme depicting the formation of a three dimensional nano-network of bridged micelles upon temperature change. At temperatures lower than the critical gelation temperature (CGT), the triblock copolymer forms a homogenous therapeutic injectable solution, held together by hydrogen bonding interaction. Above the CGT, the solution swells forming an ordered packing, with hydrophobic forces dominating the three dimensional nano-network of bridged micelles.

FIG. 2 is a graph showing sol-gel phase transition at different LA/GA ratios for polymer solutions having PEG MW 1500 Da. As the LA/GA ratio increased, the gelation temperature decreased. Comparing the system having a LA/GA ratio of 1/1 with the system having a ratio of 15/1 at 10 (w/v)%, the gelation temperature decreased from 42° C. to 35° C.

FIGS. 3A-3B are graphs illustrating the finding that systems having lower PEG MW (1000 D) have decreased gelation temperature. For systems with shorter PEG MW, the gelation temperature decreased below room temperature at the different solution concentrations due to a close packing among micelles. For all systems, the gelation temperature was not sensitive to changes in solution concentration to a statistically relevant degree.

FIG. 4 is a graph showing percentage of visible light transmittance through the self-assembled hydrogels according to embodiments of the invention. At high LA/GA ratios (15/1), as the polymer solution concentration increased, the percentage of visible light passing through the hydrogel increased.

FIG. 5 is a graph showing in vitro release of dexamethasone from PLGA-PEG-PLGA hydrogel at 25 (w/v)% polymer concentration. In 8 days, 88% of the total amount of loaded drug was released from the system at a rate of 0.677±0.035 μg/hr during the first 100 hours, and thereafter the release rate was 0.376±0.002 μg/hr.

FIG. 6 is a graph showing in vitro release of FITC-dextran from PLGA-PEG-PLGA hydrogel at 25 (w/v)% polymer concentration. In 5 days, about 52% of the total amount loaded was released from the system at a rate of 0.84±0.14 μg/hr for the first 55 hours, and thereafter at a rate of 0.23±0.02 μg/hr.

FIG. 7 is a graph showing in vitro release of nucleic acid conjugates from PLGA-PEG-PLGA hydrogel at 25 (/wv)%. In 20 days, 34% was released from the system with 31 g of nucleic acid conjugates at a rate of 0.028±0.004 μg/hr for the first 363 hours, thereafter at a rate of 0.020±0.002 μg/hr.

FIG. 8 is a graph showing in vitro release of Hyaluronic acid (HA) from PLGA-PEG-PLGA-PEG hydrogels at 25 (w/v)% modified with poly(L-Lysine) (PLL). In 10 days, 87% (13 μg HA) of the total amount loaded (15 μg HA) was released from the control systems (no PLL) at a rate of 0.087±0.044 μg/hr. In addition, 73% (11 μg HA) of the total amount loaded (15 μg HA) was released from the PLGA-PEG-PLGA systems modified with 10 (w/v)% PLL at a rate of 0.055±0.026.

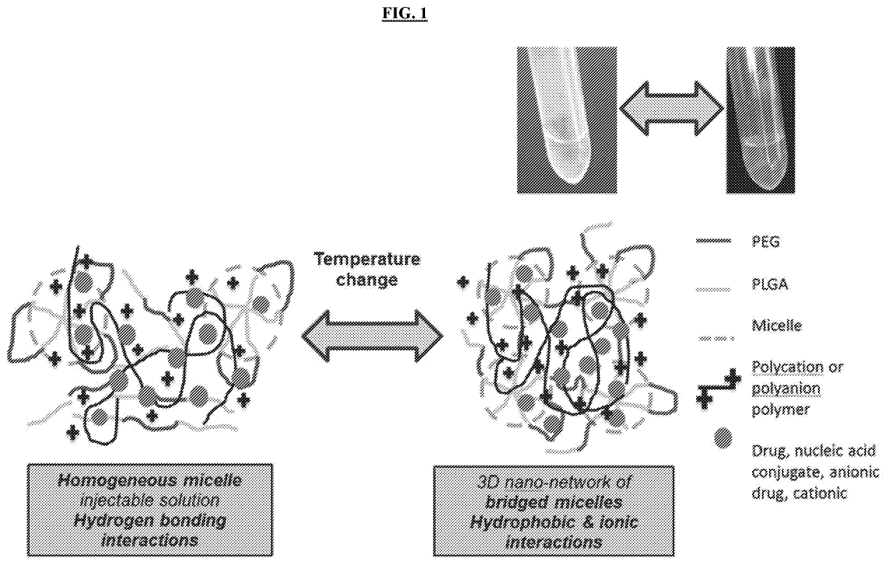

### DETAILED DESCRIPTION OF THE INVENTION

In one aspect, the present invention relates to thermo-reversible hydrogel drug delivery compositions comprising at least one biodegradable copolymer drug carrier. In certain embodiments, the hydrogel compositions of the invention are optically clear at body temperature, and are suitable for use in local delivery of ocular therapeutics. In other embodiments, the hydrogel compositions of the invention provides a means for sustained and extended drug delivery to the eye. In yet other embodiments, the hydrogel composition of the invention can be used to treat PCO. In another aspect, the invention provides methods of treating an eye disease in a subject in need thereof, the method comprising administering a hydrogel composition of the invention to the eye of the subject. The invention should not be construed to be limited to delivery of ocular therapeutics, at least because the compositions of the invention can be used to provide sustained drug delivery to any body part of a mammal, such as a human. Non-ionic polymer systems, such as PLGA-PEG-PLGA triblock copolymers, exhibit a reversible sol-gel-sol phase transition with increasing temperature that occurs through reverse gelation chemistry. The sol-gel transition, also known as gelation, is characterized by the point at which the solution transforms into a physical hydrogel. This point is known as the critical gelation temperature (CGT). Critical gelation temperature is one of the most important parameters in self-assembled systems used as potential injectable drug delivery vehicles. The sol-gel transition temperature, or CGT, can be tailored and controlled based on the desired applications by varying the following triblock copolymer properties: LA/GA ratio, molecular weight of block segments (PEG MW and PLGA MW), triblock copolymer MW, polydispersity index (PDI), addition of other molecules in the copolymer formulation (drugs or salts), chemical composition of blocks, end group functionality, and polymer solution concentration.

In one aspect, the present invention is directed to drug delivery compositions that convert to an in-situ forming hydrogel (in a non-limiting example, at body temperature) comprising a carrier composition containing a biocompatible polymer of at least one A-B-A triblock copolymers, wherein the A block is a biodegradable polyester or poly(ortho ester) and the B block is polyethylene glycol (PEG) at a concentration from about 1 to about 99 weight percent based on the total weight of the composition. In certain embodiments, the composition further contains a biocompatible solvent in a sufficient amount to solubilize the biocompatible polymer (thus forming a homogenous injectable solution) at temperatures below the CGT of the copolymer. In other embodiments, at temperatures above CGT, the composition of the invention swells and forms a transparent three dimensional nano-network. In yet other embodiments, the compositions of the invention in a gel state have at least about 50%, about 75%, about 90%, about 95%, about 98%, about 99%, or about 100% transmittance to visible light. In yet other embodiments, the compositions of the invention in a gel state have at least about 90% transmittance to wavelengths within about 390-780 nm, 400-780 nm, 410-780 nm, 420-780 nm, 430-780 nm, 440-780 nm, 450-780 nm, 460-780 nm, 470-780 nm, 480-780 nm, 490-780 nm, 500-780 nm, 510-780 nm, 520-780 nm, 530-780 nm, 540-780 nm, 550-780 nm, 560-780 nm, 570-780 nm, 580-780 nm, 590-780 nm, and/or 600-780 nm.

In certain embodiments, A is a PLGA unit and B is a PEG unit. In other embodiments, the linkage between PLGA and PEG monomeric units is via an ester linkage.

### Compositions

In one aspect, the invention provides a hydrogel composition comprising a biodegradable copolymer. In certain embodiments, the copolymer comprises an A-B-A block structure. In other embodiments, the A block is at least one selected from the group consisting of poly(D,L-lactic-co-glycolic acid) (PLGA), poly(propylene oxide) (PPO), poly(dioxanone) (PDS), and poly(L-lactic acid-co-caprolactone) (PLLACL). In yet other embodiments, the B block is at least one selected from the group consisting of polyethylene glycol (PEG), poly(vinyl alcohol) (PVA), hydroxypropyl methylcellulose (HPMC), poly(2-hydroxyethyl methacrylate) (polyHEMA), chitosan, and methoxy poly(ethylene glycol) (MPEG). In yet other embodiments, the A block is poly(D,L-lactic-co-glycolic acid) (also referred to poly(DL-lactic-co-glycolic acid) or PLGA). In yet other embodiments, the A block is poly(propylene oxide) (PPO). In yet other embodiments, the A block is poly(dioxanone) (PDS). In yet other embodiments, the A block is poly(L-lactic acid-co-caprolactone) (PLLACL). In yet other embodiments, the B block is polyethylene glycol (PEG). In yet other embodiments, the B block is poly(vinyl alcohol) (PVA). In yet other embodiments, the B block is hydroxypropyl methylcellulose (HPMC). In yet other embodiments, the B block is poly(2-hydroxyethyl methacrylate) (polyHEMA). In yet other embodiments, the B block is chitosan. In yet other embodiments, the B block is methoxy poly(ethylene glycol) (MPEG). In yet other embodiments, the A block is poly(D,L-lactic-co-glycolic acid) (PLGA) and the B block is polyethylene glycol (PEG). In yet other embodiments, the composition further comprises at least one pharmaceutical agent. In yet other embodiments, the composition further comprises at least one pharmaceutically acceptable carrier.

In certain embodiments, the biodegradable copolymer has an average molecular weight (MW) of about 500 to about 20,000 Daltons. In other embodiments, the biodegradable copolymer has an average molecular weight of about 1,000 to about 10,000 Daltons. In yet other embodiments, the biodegradable copolymer has an average molecular weight of about 1,000 to about 5,000 Daltons.

In certain embodiments, the PEG component of the copolymer has an average molecular weight (MW) of about 500 Da to about 2500 Da, or about 1000 Da to about 1750 Da. In other embodiments, the PEG average MW is about 1000 Da. In yet other embodiments, the PEG average MW is about 1500 Da.

In certain embodiments, the weight ratio of PLGA to PEG (PLGA wt/PEG wt) is about 1/1 to about 20/1. In other embodiments, the PLGA/PEG weight ratio is about 1/1 to about 3.5/1. In yet other embodiments, the PLGA/PEG weight ratio is about 2/1 to about 2.3/1. In certain embodiments, the PLGA/PEG weight ratio is at least one selected from the group consisting of about 20/1, 15/1, 10/1, 5/1, 3/1, 2.3/1, 2/1, 1.5/1 and 1/1.

In certain embodiments, the PLGA component of the copolymer comprises a (D,L)-lactic acid/glycolic acid (LA/GA) molar ratio of about 1/1 to about 35/1. In other embodiments, the LA/GA molar ratio is about 1/1 to about 15/1. In yet other embodiments, the LA/GA molar ratios is at least one selected from the group consisting of about 35/1, 25/1, 20/1, 15/1, 12.5/1, 10/1, 7.5/1, 6/1, 5/1, 3/1, 2.5/1, 2/1, 1.5/1 and 1/1.

In certain embodiments, the PLGA component of the copolymer has an average MW of about 750 Da to about 2500 Da, or about 1000 Da to about 1750 Da. In other embodiments, the PLGA average MW is about 1000 Da. In yet other embodiments, the PLGA average MW is 1500 Da.

In certain embodiments, the biodegradable copolymer has a polydispersity index of about 1.2 to about 2.0.

In certain embodiments, the pharmaceutically acceptable carrier is a pharmaceutically acceptable solvent. In other embodiments, the solvent is a saline solution. In yet other embodiments, the solvent is a phosphate buffered saline solution. In certain embodiments, the concentration of biodegradable copolymer in the composition is about 5 mg/μL to about 30 mg/μL. In other embodiments, the concentration of the biodegradable copolymer in the composition is selected from the group consisting of about 5 mg/μL, 10 mg/μL, 15 mg/μL, 20 mg/μL, 25 mg/μL, and 30 mg/μL.

In certain embodiments, the hydrogel composition further comprises at least one multivalent polyion. In other embodiments, the at least one multivalent polyion has an average MW of about 500 Da to about 1,000,000 Da. In yet other embodiments, the at least one multivalent polyion has an average MW of about 15,000 Da to about 300,000 Da. In yet other embodiments, the at least one multivalent polyion has an average MW of about 15,000 Da to about 30,000 Da. In yet other embodiments, the concentration of the at least one multivalent polyion in the biodegradable copolymer composition is about 0.1 mg/μL to about 150 mg/μL. In yet other embodiments, the concentration of the at least one multivalent polyion in the biodegradable copolymer composition is selected from the group consisting of 0.1, 0.5, 1, 5, 10, 30, 50, 70, 90, 100, 150, 200 and 250 mg/μL. In yet other embodiments, the multivalent polyion is a polycation or a polyanion. In yet other embodiments, the multivalent polycation is at least one selected from the group consisting of poly(L-Lysine) (PLL), polyethylenimine (PEI), poly[α-aminobutyl)-1-glycolic acid] (PAGA), poly(β-amino esters) (PBAEs), Polydiallyldimethylammonium chloride (polyDADMAC), and chitosan. In yet other embodiments, the multivalent polycation is poly(L-Lysine) (PLL). In yet other embodiments, the multivalent polycation is polyethylenimine (PEI). In yet other embodiments, the multivalent polycation is poly[α-aminobutyl)-1-glycolic acid] (PAGA). In yet other embodiments, the multivalent polycation is poly(β-amino esters) (PBAEs). In yet other embodiments, the multivalent polycation is Polydiallyldimethylammonium chloride (polyDADMAC). In yet other embodiments, the multivalent polycation is chitosan. In yet other embodiments, the multivalent polyanion is at least one selected from the group consisting of poly(glutamic acid) (PGA), hyaluronic acid (HA), poly(alkyl cyanoacrylate), and poly(acrylic acid) (PAA). In yet other embodiments, the multivalent polyanion is poly(glutamic acid) (PGA). In yet other embodiments, the multivalent polyanion is hyaluronic acid (HA). In yet other embodiments, the multivalent polyanion is poly(alkyl cyanoacrylate). In yet other embodiments, the multivalent polyanion is poly(acrylic acid) (PAA).

In certain embodiments, the hydrogel composition of the invention has a gelation temperature (GT) above room temperature (about 20° C. to about 25° C.). In other embodiments, the hydrogel composition of the invention has a GT at around 30° C. to about 45° C. In other embodiments, the hydrogel composition of the invention has a GT at around 32° C. to about 37° C. In other embodiments, the hydrogel composition of the invention has a GT at or around the body temperature of a mammal of interest. In yet other embodiments, the hydrogel composition of the invention has a GT of about 34-37° C.

In certain embodiments, the at least one pharmaceutical agent is hydrophobic. In other embodiments, the at least one pharmaceutical agent is hydrophilic.

In certain embodiments, the at least one pharmaceutical agent is selected from the group consisting of an antimicrobial agent, an antibiotic, an anti-inflammatory agent, a corticosteroid, an SAID, an NSAID, an immunosuppressive agent (such as, but not limited to cyclosporine), an immune-modulating agent, an apoptosis inducing agent, an anti-cancer agent, a cycloplegic agent, a mydriatic agent, a comfort agent, a lubricating agent, an anti-glaucoma agent, an anti-allergy agent, a cytotoxic agent, an anti-TNF agent, a collagen, a gamma-globulin, an interferon, a vasoconstrictor agent, a vasodilation agent, a platelet activator factor antagonist, a fibrinolytic agent (such as tissue plasminogen activator (tPA), streptokinase (SK), or urokinase (UK)), an anesthetic agent, a numbing agent, a nitric oxide synthase inhibitor, an antifungal agent, an antiviral agent, an antiprotozoal agent, a hemolytic, an antiparasitic agent, an antibody, a protein, a carbohydrate, a DNA segment, an RNA segment, or any combinations thereof.

In certain embodiments, the at least one pharmaceutical agent is a local anesthetic, such as but not limited to, lidocaine, bupivacaine, procaine, prilocaine, mepivaaine, dibucaine, levobupivaine, and/or cocaine.

In certain embodiments, the pharmaceutical agent is at least one selected from the group consisting of doxorubicin, hyaluronic acid, fluorescein isothyocyanate-hyaluronic acid, fluorescein isothiocyanate-dextran, dextran, hydroxypropoylemethycellulose, cyclosporine, beta-methasone, loteprednol etabonate, ketotifen fumarate, levocabastine (either as a salt or free base), latanoprost, dexamethasone, dexamethasone (as a salt, such as for example sodium phosphate), diclofenac (as a free acid or a salt, such as for example a sodium salt), moxifloxacin, trehalose, prednisolone, ibuprofen, nucleic acid nanospheres, nucleic acid conjugates, nucleic acid drug conjugates, nucleic acid drug conjugate antibodies, and any salts or solvates thereof.

In certain embodiments, the pharmaceutical agent is an anti-VEGF compound such as, but not limited to lapatinib, sunitinib, sorafenib, axitinib, cediranib, ranibizumab, pegaptanib, and/or pazopanib.

In certain embodiments, the pharmaceutical agent can be any physiologically or pharmacologically active substance or substances optionally in combination with pharmaceutically acceptable carriers and additional ingredients such as antioxidants, stabilizing agents, antibodies, permeation enhancers, and so forth, which do not substantially adversely affect the advantageous results that can be attained. These agents can further include vitamins, nutrients, or the like.

In certain embodiments, the hydrogel composition is a flowable liquid solution below the GT. In other embodiments, below the GT, the hydrogel composition is a flowable solution having a viscosity that allows for it be formulated as an injectable pharmaceutical composition.

In certain embodiments, above the GT, the hydrogel composition forms an orderly packed, three-dimensional hydrogel. In other embodiments, the hydrogel is a substantially transparent gel. In yet other embodiments, the hydrogel has a visible light transparency of at least 50%, 55%, 60%, 65%, 70%, 75%, 80%, 85%, 90%, 95%, 96%, 97%, 98%, 99%, or 100%. In other embodiments, the hydrogel has a visible light transparency of about 50% to visible light from 390 to 500 nm, and at least 75% to visible light from 500-780 nm. In yet other embodiments, the hydrogel formed in situ has a visible light transparency of at least 75%. In yet other embodiments, the hydrogel prevents transmittance of at least a fraction of ultraviolet light (and/or a fraction of a range thereof).

In certain embodiments, the hydrogel composition is capable of sustained release of at least 50% of the initial loaded amount of the at least one pharmaceutical agent over an extended period of time. In other embodiments, the extended period of time is at least one selected from the group consisting of 1 day, 3 days, 7 days, 2 weeks, 1 month, 2 months, 3 months, 4 months, 6 months, 1 year and any periods of time in-between. In other embodiments, the hydrogel composition exhibits sustained and controlled polymer degradation, thereby facilitating drug release kinetics for over days, weeks, or months.

In certain embodiments wherein the hydrogel composition further comprises at least one multivalent polycation, the sustained release of the at least one pharmaceutical agent is at least in part due to complexation of the at least one pharmaceutical agent by the at least one multivalent polycation. In other embodiments, the multivalent polycation is present in the composition at a concentration of about 0.1 mg/μL to about 150 mg/μL. In other embodiments, the at least one pharmaceutical agent comprises at least one anionic moiety, allowing for ionic complexation with the at least one multivalent polycation.

A non-limiting advantage to the compositions of the invention lies in their ability to form a transparent hydrogel after administration into an ocular target. As such, any combination of pharmaceutical agents and copolymer system that provides at least a 50% visibility threshold when in situ should be understood to be part of the present invention.

In certain embodiments, the hydrogel composition further comprises at least one visualization agent. In other embodiments, the visualization agent is any compound or material additive that allows for the hydrogel composition to be visually identified and distinguished from a surrounding material. In yet other embodiments, exemplary visualization agents include, but are not necessarily limited to a fluorescent agent, a coloring agent and a reflective agent. In yet other embodiments, the visualization agent is a biodegradable visualization agent. In yet other embodiments, the visualization agent is at least one compound selected from the group consisting of 1-(1-carboxyethyl)-2,6-dioxi-1,2,3,6-tetrahydropyridine-4-carboxylic acid, BPLP-cystein (BPLP-Cys), and BPLP-serine. In yet other embodiments, the visualization agent is at least one biodegradable photo-luminescent polymer disclosed in U.S. Pat. No. 8,530,611, which is incorporated herein in its entirety by reference. In yet other embodiments, the visualization agent is an aliphatic biodegradable photoluminescent polymer (BPLP) composition comprising: a degradable oligomer, wherein the oligomer is synthesized from a biocompatible multifunctional carboxylic acid comprising a hydroxyl group, a diol, and an amino acid; wherein the amino acid is linked as a side group to the degradable oligomer backbone; wherein fluorescence emanates from a 6-membered ring formed by a carboxylic acid group of the amino acid, an alpha carbon of the amino acid, an amide linkage formed by an amino group of the amino acid, and a central carbon of the multifunctional carboxylic acid via an esterification reaction of the carboxylic acid group of the amino acid and the hydroxyl group of the multifunctional carboxylic acid. In yet other embodiments, the biocompatible multifunctional carboxylic acid comprises citric acid, the diol comprises 1,8-octanediol, and the amino acid comprises cysteine or serine. In yet other embodiments, the diol comprises a saturated aliphatic diol, C₃-C₁₂ diol, hydrophilic diol, hydrophobic diol or any combination thereof. In yet other embodiments, the diol is selected from a 1,8-octanediol, ethylene glycol, propylene glycol, poly(ethylene glycol), poly(propylene glycol), 1,3-propanediol, ethanediol, and cis-1,2-cyclohexanediol. In yet other embodiments, the BPLP is crosslinked. In yet other embodiments, the crosslinking is achieved by radical polymerization initiated by photoinitiators or redox initiators. In yet other embodiments, the crosslinking is achieved by a condensation reaction. In yet other embodiments, an acid anhydride or a multifunctional acid chloride is used in addition to the multifunctional carboxylic acid. In yet other embodiments, the visualization agent can aid a medical professional in locating the hydrogel composition during a medical procedure.

### Methods

In another aspect, the invention provides methods of treating or preventing a disease or disorder in a subject in need thereof, the method comprising administering to a body part of the subject a therapeutically effective amount of a hydrogel composition of the invention. In certain embodiments, the hydrogel composition of the invention comprises a pharmaceutical agent capable of treating or preventing the disease or disorder in the body part of the subject. In other embodiments, the body part comprises the eye.

In certain embodiments, the method comprises administering to the subject a hydrogel composition of the invention. In other embodiments, before administration, the hydrogel composition is at a temperature below the gelation temperature and is therefore in a flowable liquid form. In yet other embodiments, after administration, the hydrogel composition warms due to contact with the body part of the subject, rises to a temperature above the gelation temperature, undergoing thermo-reversible gelation, thereby forming a hydrogel.

In certain embodiments, the hydrogel composition is administered to the subject as a flowable liquid by injection, such that after injection, the hydrogel composition rises above the gelation temperature and forms a hydrogel in situ. In certain embodiments, the hydrogel formed in situ has a visible light transparency of at least 50%, 55%, 60%, 65%, 70%, 75%, 80%, 85%, 90%, 95%, 96%, 97%, 98%, 99%, or 100%. In other embodiments, the hydrogel formed in situ has a visible light transparency of about 50% to visible light from 390 to 500 nm, and at least 75% to visible light from 500-780 nm. In yet other embodiments, the hydrogel formed in situ has a visible light transparency of at least 75%.

In certain embodiments, the disease or disorder is a disease or disorder of the eye. In other embodiments, the disease or disorder is at least one selected from the group consisting of posterior capsule opacification (PCO), age-related macular degeneration (AMD), diabetic eye disease, diabetic macular edema (DME), macular edema, uveitis, glaucoma, Behcet's Disease (Adamantiades-Behcet's disease), blepharospasm, corneal diseases, retinal diseases, dry eye diseases, eye inflammation and eye infection.

In certain embodiments, invention provides a method of treating post-surgical corneal trauma in a subject in need thereof, the method comprising administering to the subject a composition of the invention. In other embodiments, the method can be used to prevent infection or inflammation after primary eye surgery. In other embodiments, the primary surgery is a surgery to treat one or more eye diseases or disorders. In yet other embodiments, the primary surgery is cataracts surgery.

In other embodiments, the hydrogel composition is administered to the subject via intraocular injection.

In certain embodiments, the subject is a mammal. In other embodiments, the subject is a human.

### Combination and Concurrent Therapies

In one embodiment, the compositions of the invention are useful in the methods of present invention when used concurrently with at least one additional compound useful for preventing and/or treating diseases and/or disorders contemplated herein.

In one embodiment, the compositions of the invention are useful in the methods of present invention in combination with at least one additional compound useful for preventing and/or treating diseases and/or disorders contemplated herein.

These additional compounds may comprise compounds of the present invention or other compounds, such as commercially available compounds, known to treat, prevent, or reduce the symptoms of diseases and/or disorders contemplated herein. In certain embodiments, the combination of at least one compound of the invention or a salt thereof, and at least one additional compound useful for preventing and/or treating diseases and/or disorders contemplated herein, has additive, complementary or synergistic effects in the prevention and/or treatment of diseases and/or disorders contemplated herein.

As used herein, combination of two or more compounds may refer to a composition wherein the individual compounds are physically mixed or wherein the individual compounds are physically separated. A combination therapy encompasses administering the components separately to produce the desired additive, complementary or synergistic effects.

In certain embodiments, the compound and the agent are physically mixed in the composition. In other embodiments, the compound and the agent are physically separated in the composition.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326), the median-effect equation (Chou & Talalay, 1984, Adv. Enzyme Regul. 22: 27-55), and through the use of isobolograms (Tallarida & Raffa, 1996, Life Sci. 58: 23-28). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The therapeutically effective amount or dose of a compound of the present invention depends on the age, sex and weight of the patient, the current medical condition of the patient and the progression of a disease or disorder contemplated in the invention.

A medical doctor, e.g., physician or veterinarian, having ordinary skill in the art may readily determine and prescribe the effective amount of the pharmaceutical composition required.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage, for example from once a month, or once every 6 months, or to once a year. When single dosages are used, the amount of each dosage may be the same or different.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 300 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the compositions of the invention are administered to the patient in dosages that range from once a week, once a month, once every year, two years, or three years. In another embodiment, the compositions of the invention are administered to the patient in range of dosages that include, but are not limited to, once every week, once every month, every two months, every three months to once a year, once every two years, and once every three years. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 7 days and 1 year, including by way of example only, 7 days, 15 days, 30 days, 60 days, 90 days, 180 days, 365 days, 730 days, or 1,095 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In one embodiment, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single dose (e.g., about every once a week to about every 3 years). When single doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the compositions of the invention are formulated using at least one pharmaceutically acceptable excipients or carriers. In one embodiment, the pharmaceutical compositions of the invention comprise a therapeutically effective amount of a compound of the invention and a pharmaceutically acceptable carrier.

The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce at least one symptoms of a disease or disorder contemplated in the invention.

Formulations may be employed in admixtures with conventional excipients, i.e., pharmaceutically acceptable organic or inorganic carrier substances suitable for any suitable mode of administration, known to the art. The pharmaceutical preparations may be sterilized and, if desired, mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., analgesic agents.

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, inhalation, and topical administration. In certain embodiments, routes of administration of any of the compositions of the invention include nasal, inhalational, intratracheal, intrapulmonary, and intrabronchial. In preferred embodiments, the composition of the invention is administered via intraocular injection.

Suitable compositions and dosage forms include, for example, dispersions, suspensions, solutions, syrups, granules, beads, powders, pellets, liquid sprays for nasal or oral administration, dry powder or aerosolized formulations for inhalation, and the like. It should be understood that the formulations and compositions that would be useful in the present invention are not limited to the particular formulations and compositions that are described herein.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### Definitions

As used herein, each of the following terms has the meaning associated with it in this section.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

Generally, the nomenclature used herein and the laboratory procedures in tissue engineering and biomaterial science are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, “biodegradable” means that the block copolymer or oligomer can chemically break down or degrade within the body to form nontoxic components. The rate of degradation can be the same or different from the rate of drug release and can be different for each product formed via hydrolysis, enzymatic breakdown, or other forms of degradation.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary and topical administration.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “gel” or “hydrogel” refers to a three-dimensional polymeric structure that itself is insoluble in a particular liquid but which is capable of absorbing and retaining large quantities of the liquid to form a stable, often soft and pliable, but always to one degree or another shape-retentive, structure. When the liquid is water, the gel is referred to as a hydrogel. Unless expressly stated otherwise, the term “gel” will be used throughout this application to refer both to polymeric structures that have absorbed a liquid other than water and to polymeric structures that have absorbed water, it being readily apparent to those skilled in the art from the context whether the polymeric structure is simply a “gel” or a “hydrogel.”

The terms “patient,” “subject” or “individual” are used interchangeably herein, and refer to any animal, or cells thereof whether in vitro or in situ, amenable to the methods described herein. In a non-limiting embodiment, the patient, subject or individual is a human.

As used herein, “PLGA” refers to a copolymer or copolymer radicals derived from the condensation copolymerization of lactic acid and glycolic acid, or, by the ring opening copolymerization of lactide and glycolide. The terms lactic acid and lactate are used interchangeably; glycolic acid and glycolate are also used interchangeably.

As used herein, “PLA” refers to a polymer derived from the condensation of lactic acid (LA) or by the ring opening polymerization of lactide.

As used herein, “PGA” refers to a polymer derived from the condensation of glycolic acid (GA) or by the ring opening polymerization of glycolide.

As used herein, “PEG” or “POE” refers to a hydrophilic polymer derived from ethylene oxide.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

As used herein, the term “thermo-reversible gelation” is the phenomenon whereby an aqueous solution of a block copolymer spontaneously increases in viscosity, and in many instances transforms into a semisolid gel, as the temperature of the polymer solution is increased above the gelation temperature of the block copolymer solution. For the purpose of the invention, the term gel includes both the semisolid gel state and the high viscosity state that exists above the gelation temperature. When cooled below the gelation temperature, the gel spontaneously reverses to reform the lower viscosity polymer solution.

As used herein, the terms “% (w/v)” or “(w/v)%” refer to a percentage derived by dividing the mass of the polymer additive in milligram (mg) by the volume of the solution in microliters (L). As used herein, these terms can be used interchangeably with “mg/μL”.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Materials

Triblock copolymer, PLGA-PEG-PLGA, with varying LA/GA ratios, PLGA/PEG ratios, PEG MW, total molecular weight, total number average molecular weight (MN), and polydispersity index were obtained from PolyScitech, Inc (West Lafayette, Ind.). Phosphate Buffer Saline and FITC-Dextran (150 KDa), Fluorescein-Hyaluronic acid (800 KDa), and Poly(L-Lysine) (15-30 KDa) were obtained from Millipore Sigma (St. Louis, Mo.) and were used as provided. Dexamethasone, 98% was obtained from Alfa Aesar and was used as is. Nucleic acid conjugates were obtained from Genisphere LLC (Hatfield, Pa.) and was used as provided. 30 mL syringes with BD Luer-Lok® Tip were obtained from BD Medical. SpectraMax M3 Series Multi-Mode Microplate reader from Molecular Devices, LLC (Sunnyvale, Calif.).

### Hydrogel Formation

Triblock copolymer, PLGA-PEG-PLGA, was dissolved in Phosphate Buffer Saline to make the following concentrations: 10, 15, 20, and 25 w/v % (samples B1-B4, Table 1). The solutions of samples B1-B4 were mixed at room temperature for a period of 24 hours, or until all of the polymer was dissolved. The solutions of samples C1-C3 (Table 1) were mixed at 5° C. for a period of 24 hours. All solutions were stored at 5° C. for a minimum of 24 hours before testing.

### Gelation Temperature Characterization

The sol (flow) or gel (no-flow) phase transition temperatures of the triblock copolymers in PBS at varied concentrations were determined by the vial-inversion method. A total of 0.5 mL of hydrogel solution was added into a 2 mL vial. The vials were immersed in a water bath. The temperature was adjusted between 1 and 50° C., and the vials were allowed to equilibrate for 20 minutes at each temperature. If no flow was observed within 30 seconds of inverting the vial, the triblock copolymer at such temperature and concentration was considered a gel.

### Light Transmittance

The triblock copolymer solution at different concentrations (10-25 w/v %) of samples B1-B4 (Table 1) were used to test light transmittance. Samples of 200 μL were placed on a 96-well plate. An absorbance scan was performed from 250 nm to 850 nm using the UV-Spectroscopy, Infinite 200 PRO NanoQuant Microplate Reader (Tecan). Wavelength was plotted against percentage of light transmittance.

### In Vitro Drug Release

The triblock copolymer solution with LA/GA ratio of 15/1 at different concentrations (14-25 w/v %) was mixed with either Dexamethasone, FITC-Dextran, Fluorescein-Hyaluronic acid, and nucleic acid conjugates. The final volume of the hydrogel solution with the compound of interest was constrained to 100 μL because of the anatomical space in the lens capsule. Physiological flow rate (2.4 μl/min) was achieved using microfluidic devices. The devices resemble the anatomical dimensions of both the anterior and posterior chamber of the human eye, as well as the physiological dynamics of the aqueous humor flow. All of the release studies were conducted at 35° C.
a. Dexamethasone (Hydrophobic): 100 μL of PLGA-PEG-PLGA hydrogel solution at 25 (w/v) % was mixed with 100 μg of dexamethasone. The collected samples were analyzed via HPLC.
b. FITC-Dextran (Hydrophilic): 100 μL of PLGA-PEG-PLGA polymer solution at 25 (w/v) % was mixed with 100 μg of FITC-Dextran (150 KDa). The collected samples were analyzed via fluoresce spectroscopy at an excitation, filter, and emission wavelengths (nm) of 483, 515 524, respectively
c. Nucleic acid conjugates (Hydrophilic): 62 μL of nucleic acid conjugates in PBS solution (31 μg) were mixed with 38 μL of hydrogel solution at a concentration of 25 (w/v) %. Because the nucleic acid conjugates were already dissolved in PBS, the hydrogel solution was diluted down to 14 (w/v) %. The collected samples were analyzed via fluorescence spectroscopy at an excitation, filter, and emission wavelengths (nm) of 642, 665, and 670, respectively.
d. Hyaluronic acid (Hydrophilic): 30 μL of hyaluronic acid (HA, 800 KDa) stock solution at a concentration of 0.5 mg/mL in PBS (15 ug of HA) was mixed with 56 μL of hydrogel solution at a concentration of 25 (w/v)%. In addition, 14 μL of PBS were added to diluted the hydrogel solution down to 14 (w/v)%. Samples containing PLL were prepared by adding to the already mentioned composition 10 mg of PLL (10 (w/v)% PLL). The collected samples were analyzed via fluorescence spectroscopy at an excitation, filter, and emission wavelengths (nm) of 483, 495, 518, respectively.

### Example 1: Hydrogel Formation and Gelation Temperature Characterization

PLGA-PEG-PLGA triblock copolymers were obtained with different parameters, as listed in Table 1. The triblock copolymer molecular weight (MW), number average molecular weight (MN), and polydispersity index (PDI) were measured via GPC. The PLGA/PEG ratios and LA/GA ratios were measured via ¹H-NMR.

TABLE 1
Properties of PLGA-PEG-PLGA Triblock Copolymers.

| | | PLGA/PEG | (D,L)LA/GA^{a} | | | |
|---|---|---|---|---|---|---|
| Sample | Mₙ^{a} | (wt/wt) | (mol/mol) | M_{w}^{b} | Mₙ^{b} | M_{w}^{b}/Mₙ^{b} |
| A1 | 750-1500-750 | 1.0 | 15/1 | 6396 | 4205 | 1.52 |
| A2 | 1700-1500-1700 | 2.3 | 15/1 | 6302 | 4926 | 1.28 |
| A3 | 2250-1500-2250 | 3.0 | 15/1 | 11627 | 6840 | 1.7 |
| B1** | 1450-1500-1450 | 1.9 | 1/1 | 9798 | 5691 | 1.7 |
| B2 | 1600-1500-1600 | 2.1 | 3/1 | 8220 | 5807 | 1.42 |
| B3 | 1500-1500-1500 | 2.0 | 6/1 | 6438 | 5385 | 1.20 |
| B4 | 1700-1500-1700 | 2.3 | 15/1 | 6302 | 4926 | 1.28 |
| C1 | 1000-1000-1000 | 2.0 | 1/1 | 5495 | 4365 | 1.26 |
| C2 | 1000-1000-1000 | 2.0 | 6/1 | 9721 | 5958 | 1.63 |
| C3 | 1000-1000-1000 | 2.0 | 15/1 | 7294 | 4037 | 1.81 |
| B1-1* | 1400-1500-1400 | 1.87 | 1/1 | 8529 | 4873 | 1.7 |
| B1-2* | 1500-1500-1500 | 2.0 | 1/1 | 11067 | 6508 | 1.7 |

^{a}The LA/GA molar ratios were calculated with ¹H-NMR.
^{b}The Mn and Mw values of the triblock copolymers, and their polydispersity indices (Mw/Mn) were measured via GPC.
**The PLGA Mn of (1450 Da), the Mn, and the Mw values are averages between the respective values between B1-1* and B1-2* in the form of (mean ± SD)

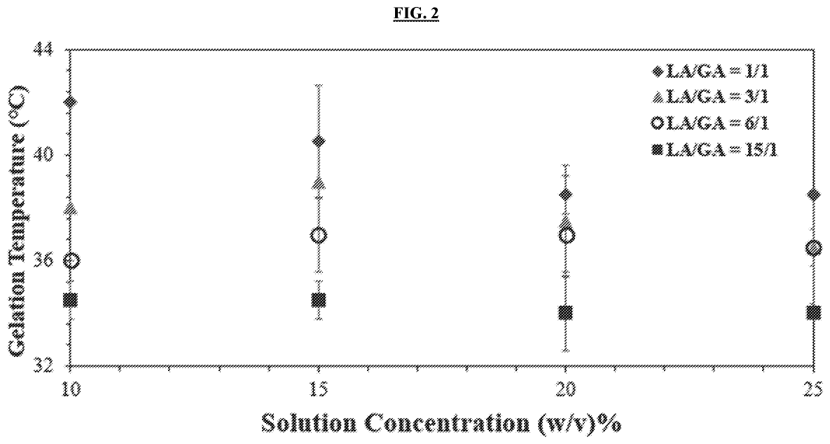

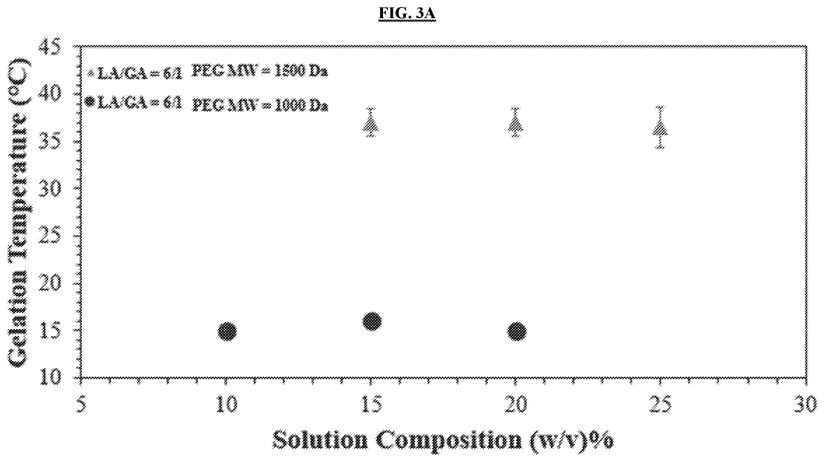

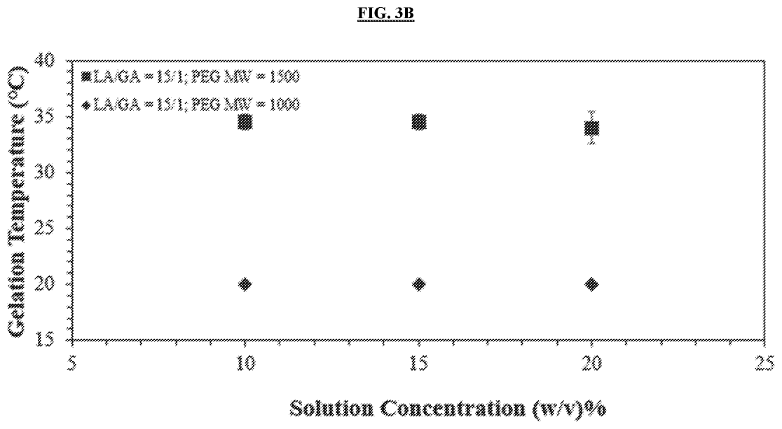

Samples B1-C3 were divided into two groups: samples B1-B4 contained PEG MW of 1500 Da, and samples C1-C3 contained PEG MW of 1000 Da. FIGS. 2 & 3A-3B show the gelation temperature in (° C.) vs solution concentration of samples B1-B4, B3 vs C2 and B4 vs C3 respectively. For each different LA/GA ratio, GT did not drastically change with solution concentration, indicating that the critical gelation temperature is not sensitive to changes in solution concentration. Further, FIG. 2 shows that with increasing LA/GA ratio or hydrophobicity, the GT decreases. FIGS. 3A-3B, however, show that as PEG MW decreases, GT drops below room temperature.

A parametric study was conducted to understand the influence that the different properties of PLGA-PEG-PLGA have on the gelation temperature. The parameters, or independent variables, of the triblock copolymer are: LA/GA ratio as well as triblock copolymer Mn, MW, and PDI. Table 2 shows the linear regression coefficients (slopes) and the correlation coefficients with their respective p-values. The Spearman Rank Correlations between “LA/GA and GT”, and “MW and GT” show that both variables influence gelation temperature overall.

TABLE 2
Linear regression coefficients and correlation
coefficients with their p-values

| P | Linear Regression Coefficient | Correlation Coefficient |
|---|---|---|
| LA/GA | −44E−3 ± 8.9E−3 (p-value <0.1) | −1 (p-value <0.05)** |
| Mn | 42E−3 ± 9.4E−3 (p-value <0.1) | +0.8 (p-value >0.05) |
| MW | 39E−3 ± 9.8E−3 (p-value <0.1) | +1 (p-value <0.05)** |
| PDI | 37E−3 ± 15E−3 (p-value <0.1) | −0.8 (p-value >0.05) |

*p-values < α = 0.1 represent linear coefficients are significantly different from zero
**p-values < α = 0.05 represent correlation coefficients to be significant between the independent variable and gelation temperature.

### Example 2: Light Transmittance

UV-Spectroscopy absorbance readings assisted in determining the visible light transmittance at constant PLGA/PEG ratios and a constant PEG MW of 1500 Da. As solution concentration increased, the percentage of light transmittance increased, creating a more optically clear hydrogel.

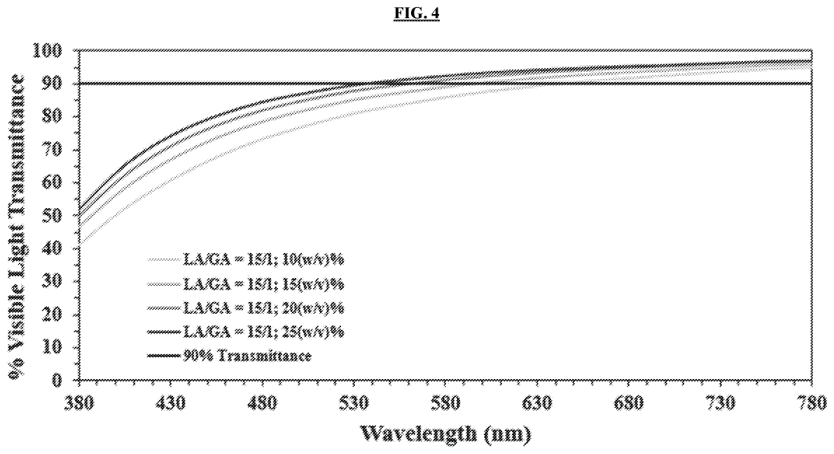

FIG. 4 shows the percentage of transmitted light through the hydrogel. At high LA/GA ratios (15/1) and high polymer solution concentrations, the percentage of light transmitted through the hydrogel was over 90. The PLGA-PEG-PLGA hydrogel system absorbs UV wavelengths, as well as shorter-wavelengths in the visible light spectrum (violet and blue). This is beneficial to the patient as it provides further protection to the retina while recovering from surgery. Blue-violet light is a risk factor for the development of age-related macular degeneration.

PLGA-PEG-PLGA hydrogels undergo two different phase transitions; sol-to-gel and gel-to-sol. The latter phase transition occurs at temperatures higher than body temperature. Therefore, such phase transition is not relevant for applications in ocular drug delivery. At room temperature the polymer solution is in liquid state. However, as temperature increases, hydrophobic forces take place and micelles start forming. At this point, the hydrogel is transparent. As temperature continues to increase closer to the second phase transition (gel-to-sol), the micelles are packed and the hydrogel becomes opaque. This material has two components, PDLA and PLLA, causing the solution to become a more amorphous gel. This allows light to be transmitted due to the disordered polymer chains.

### Example 3: In Vitro Drug Release

The release of drugs from the PLGA-PEG-PLGA system depends on diffusion and polymer degradation. The PLGA core of each micelle encapsulates the drug, creating a concentration gradient. The concentration gradient facilitates the movement of drug out of the micelles and into the localized area where it can take effect. However, over time the self-assembled polyester/polyether matrix degrades via hydrolysis of ester bonds, releasing much more drug from the nanogel as it erodes. The final degradation products from the gel are lactic acid and glycolic acid, which are excreted via the kidneys, and PEG, which enters into systemic circulation to be released. The release of various sized drugs was performed using Dexamethasone, FITC Dextran, and nucleic acid conjugates to determine if this system was capable of extended and controlled release over one week.

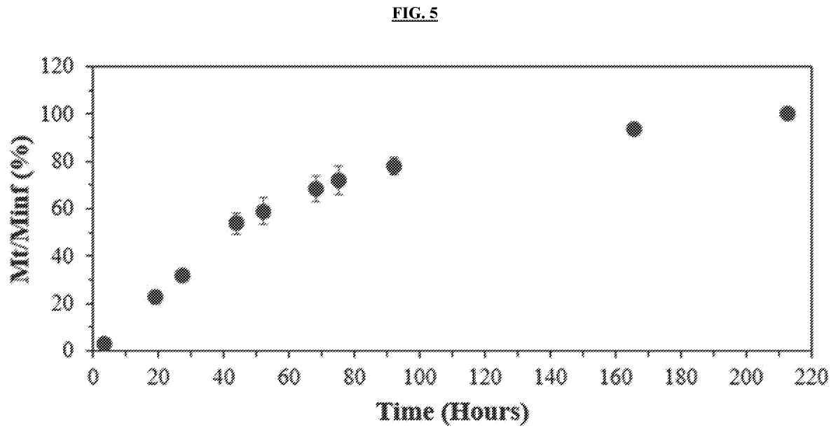

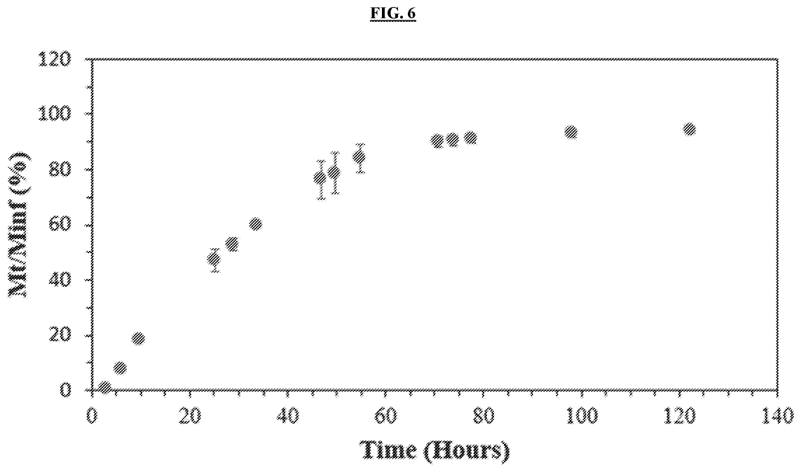

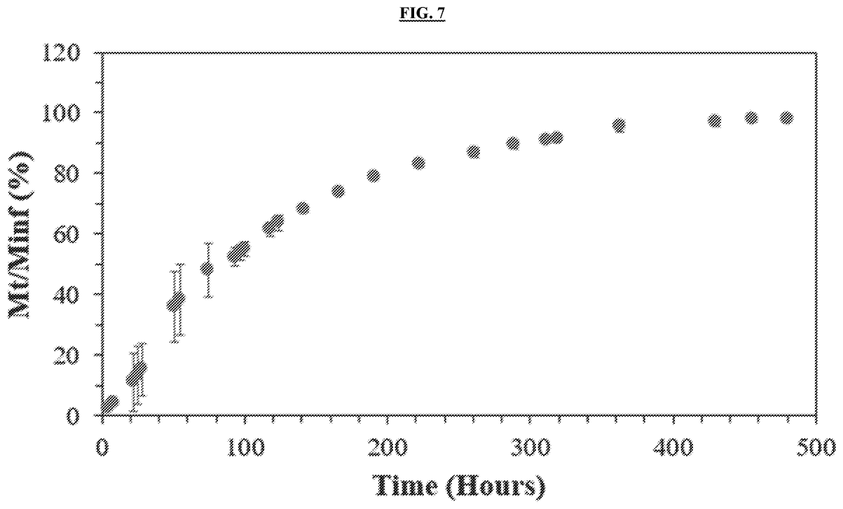

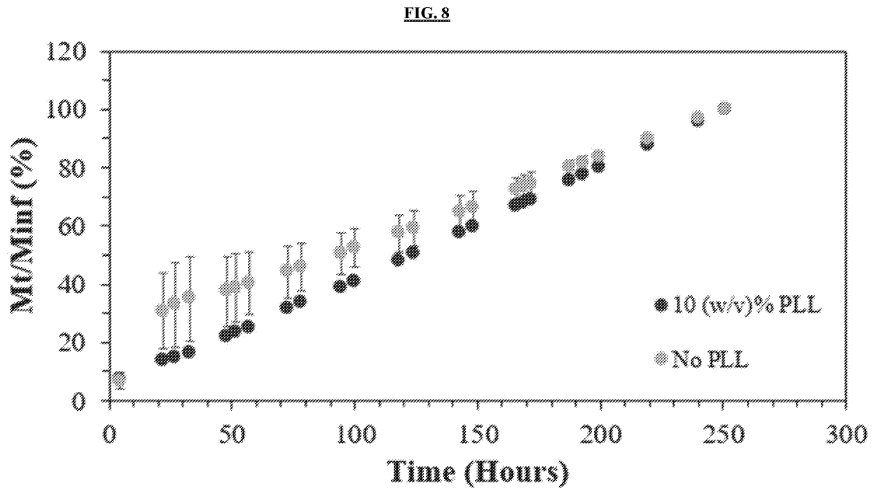

FIGS. 5-8 show the fractional release data of dexamethasone, FITC-Dextran, nucleic acid conjugates, and hyaluronic acid systems with and out PLL, respectively. Dexamethasone is a hydrophobic molecule and its hydrodynamic diameter size is 1 nm. Because of its low solubility range in water, only small amounts of dexamethasone could be mixed directly into the hydrogel solution. At the gelation temperature (35° C.), dexamethasone was encapsulated within the core of the hydrophobic PLGA micelles. The release rate of Dexamethasone from the thermosensitive hydrogel was 0.677±0.035 μg/hr for the first 100 hours, and in 212 hours (8 days), 80% of the payload was released 0.376±0.002 μg/hr.

FITC-Dextran is an anhydroglucose-based polymer with greater symmetry at high molecular weights. Without intending to be limited to any particular theory, as the polymer solution became a hydrogel, FITC-Dextran came in contact with the corona of the micelles (PEG units). The release rate as 0.84±0.14 μg/hr for the first 55 hours, and in 222 hours (5 days), about 52% of the initial payload was released at a rate of 0.23±0.02 μg/hr.

Nucleic acid conjugates are of interest because they can be used as drug nanocarriers to specifically target cells. The hydrogel system with 31 μg of negatively charged drug conjugates demonstrated a release rate of 0.028±0.004 μg/hr for the first 363 hours, and in 480 hours (20 days) about 34% of the initial payload was released at a rate of 0.020±0.002 g/hr.

Hyaluronic acid is an anionic, non-sulfated glycosaminoglycan found in connective, epithelial, and neural tissues. Without intending to be limited to any particular theory, as the polymer solution became a hydrogel, HA came in contact with the corona of the micelles (PEG units). Whereas for the systems with PLL, ionic interactions between PLL and HA led to ion pair formation arising from intermolecular polyionic complexation. In certain embodiments, complexation of the HA by PLL reduces release rate of the HA from the composition of the invention. The release rate of the systems with no PLL was 0.087±0.044 ug/hr, and in 249 hours (10 days), about 87% of the total amount loaded of HA was released. The release rate of the systems with 10 (w/v)% PLL as 0.055±0.026, and in 249 hours (10 days), about 73% of the total amount loaded of HA was released.

The molecules embedded in the PLGA-PEG-PLGA hydrogel were released via diffusion-degradation mechanisms caused by the hydrolysis of ester bonds. The hydrogel matrix demonstrated qualities making it an ideal material to be used in the ocular drug delivery field. When the hydrogel was mixed with the compound or drug of interest, the main properties, optical clarity, gelation temperature, and release over one week of hydrophilic and negatively charged molecules, were maintained.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or preventing a disease or disorder in the eye of a subject in need thereof, the method comprising administering to the subject a therapeutically effective amount of a composition comprising:
a biodegradable copolymer comprising an A-B-A block structure,
wherein the A block is at least one selected from the group consisting of poly(D,L-lactic-co-glycolic acid) (PLGA), poly(propylene oxide) (PPO), poly(dioxanone) (PDS), and poly(L-lactic acid-co-caprolactone) (PLLACL), and
wherein the B block is at least one selected from the group consisting of polyethylene glycol (PEG), poly(vinyl alcohol) (PVA), hydroxypropyl methylcellulose (HPMC), poly(2-hydroxyethyl methacrylate) (polyHEMA), chitosan, and methoxy poly(ethylene glycol) (MPEG);
at least one pharmaceutical agent; and
at least one pharmaceutically acceptable carrier;
wherein the composition has a gelation temperature (GT) of around 30° C. to about 37° C.

2. (canceled)

3. The method of claim 1, wherein the A block is PLGA and the B block is PEG.

4. (canceled)

5. The method of claim 1, wherein the weight ratio of PLGA to PEG (PLGA wt/PEG wt) is about 1/1 to about 5/1.

6-7. (canceled)

8. The method of claim 1, wherein the LA/GA molar ratio is at least one selected from the group consisting of about 35/1, 25/1, 20/1, 15/1, 12.5/1, 10/1, 7.5/1, 6/1, 5/1, 3/1, 2.5/1, 2/1, 1.5/1, and 1/1.

9. (canceled)

10. The method of claim 1, wherein the composition further comprises at least one multivalent polyion.

11. The method of claim 10, wherein the concentration of the at least one multivalent polyion in the biodegradable copolymer is about 0.1 mg/μL to about 150 mg/L.

12. (canceled)

13. The method of claim 10, wherein the at least one multivalent polyion is at least one selected from the group consisting of poly(L-Lysine) (PLL), polyethylenimine (PEI), poly[α-aminobutyl)-1-glycolic acid] (PAGA), poly(β-amino esters) (PBAEs), Polydiallyldimethylammonium chloride (polyDADMAC), chitosan, poly(glutamic acid) (PGA), hyaluronic acid (HA), poly(alkyl cyanoacrylate), and poly(acrylic acid) (PAA).

14-15. (canceled)

16. The method of claim 1, wherein the biodegradable copolymer has a polydispersity index of about 1.2 to about 2.0.

17-18. (canceled)

19. The method of claim 1, wherein the composition has a GT of around 34° C. to about 37° C.

20-21. (canceled)

22. The method of claim 1, wherein the disease or disorder is at least one selected from the group consisting of posterior capsule opacification (PCO), age-related macular degeneration (AMD), diabetic eye disease, diabetic macular edema (DME), macular edema, uveitis, glaucoma, Behcet's Disease (Adamantiades-Behcet's disease), blepharospasm, corneal diseases, retinal diseases, dry eye diseases, eye inflammation, eye infection, post-surgical trauma, eye infection, and eye inflammation.

23. The method of claim 1, wherein the composition is administered to the subject via intraocular injection.

24. The method of claim 1, wherein the composition is administered to the subject at a temperature below its GT, such that the composition is capable of being administered by injection to the subject and the composition undergoes thermo-reversible gelation to form a hydrogel after administration to the subject.

25. The method of claim 24, wherein the hydrogel formed after the composition undergoes thermo-reversible gelation transmits at least 50% of visible light.

26-29. (canceled)

30. A composition comprising:
a biodegradable copolymer comprising an A-B-A block structure, wherein the A block is at least one selected from the group consisting of (PLGA), poly(propylene oxide) (PPO), poly(dioxanone) (PDS), and poly(L-lactic acid-co-caprolactone) (PLLACL), and the B block is at least one selected from the group consisting of polyethylene glycol (PEG), poly(vinyl alcohol) (PVA), hydroxypropyl methylcellulose (HPMC), poly(2-hydroxyethyl methacrylate) (polyHEMA), chitosan, and methoxy poly(ethylene glycol) (MPEG);
at least one pharmaceutical agent; and
at least one pharmaceutically acceptable carrier;
wherein the composition has a gelation temperature (GT) of around 30° C. to about 37° C.

31. The composition of claim 30, which has a GT of around 34° C. to about 37° C.

32. The composition of claim 30, wherein the biodegradable copolymer has a polydispersity index of about 1.2 to about 2.0.

33. The composition of claim 30, wherein the biodegradable copolymer is present in the composition at a concentration of about 5% to about 30% (w/v).

34-36. (canceled)

37. The composition of claim 30, wherein at least one applies:
the A block is PLGA and the B block is PEG;
the PEG component of the copolymer has an average MW of about 500 to about 2,500 Daltons;
the weight ratio of PLGA to PEG (PLGA wt/PEG wt) is about 1/1 to about 5/1;
the PLGA component of the copolymer comprises a (D,L)-lactic acid/glycolic acid (LA/GA) molar ratio of about 1/1 to about 35/1;
the PLGA component of the copolymer has an average MW of about 1,000 to about 2500 Daltons.

38-43. (canceled)

44. The composition of claim 30, which further comprises at least one multivalent polyion.

45. The composition of claim 44, wherein the concentration of the at least one multivalent polyion in the biodegradable copolymer is about 0.1 mg/μL to about 150 mg/μL.

46. (canceled)

47. The composition of claim 44, wherein the at least one multivalent polyion is at least one selected from the group consisting of PLL, PEI, PAGA, PBAEs, polyDADMAC, chitosan, PGA, HA, poly(alkyl cyanoacrylate), and PAA.

48-49. (canceled)
